(19) United States
(12) Patent Application Publication
(10) Pub. No.: US 2021/0404891 A1
(43) Pub. Date: Dec. 30, 2021

(54) PRESSURE SENSOR, METHOD OF FABRICATING PRESSURE SENSOR, AND PRESSURE DETECTING DEVICE

(71) Applicants: BOE TECHNOLOGY GROUP CO., LTD., Beijing (CN); NATIONAL CENTER FOR NANOSCIENCE AND TECHNOLOGY, Beijing (CN)

(72) Inventors: Hongbian Li, Beijing (CN); Jidong Shi, Beijing (CN); Kairan Liu, Beijing (CN); Chunyan Ji, Beijing (CN); Xinguo Li, Beijing (CN); Wenbo Li, Beijing (CN)

(73) Assignees: BOE TECHNOLOGY GROUP CO., LTD., Beijing (CN); NATIONAL CENTER FOR NANOSCIENCE AND TECHNOLOGY, Beijing (CN)

(21) Appl. No.: 16/474,670

(22) PCT Filed: Dec. 25, 2018
(86) PCT No.: PCT/CN2018/123460

§ 371 Date: Jun. 28, 2019

(30) Foreign Application Priority Data
Apr. 28, 2018 (CN) 201810401719.4

(51) Int. Cl. G01L 1/22; A61B 5/024

(52) U.S. Cl. CPC G01L 1/2287; A61B 5/02444; A61B 5/02438; A61B 2562/166; A61B 2562/0247; A61B 2562/18; A61B 2562/12; A61B 2562/0285

(57) ABSTRACT

The present disclosure generally relates to pressure detection technology, and in particular, to a pressure sensor, a method of fabricating a pressure sensor, and a pressure detecting device. The pressure sensor may include a flexible nanopaper, and a graphene film on one side of the flexible nanopaper.

### CROSS-REFERENCE TO RELATED APPLICATIONS

This application claims benefit of the filing date of Chinese Patent Application No. 201810401719.4 filed on Apr. 28, 2018, the disclosure of which is hereby incorporated in its entirety by reference.

### TECHNICAL FIELD

The present disclosure generally relates to pressure detection technology, and in particular, to a pressure sensor, a method of fabricating a pressure sensor, and a pressure detecting device.

### BACKGROUND

Small variations in pressure may carry great significance in many situations. For example, detecting variations in pulse during medical diagnosis can be symptomatic of certain medical conditions, or detecting variations in sound can help establish human-computer interaction platform. In situations such as those, the accurate detection of the small variations in pressure is crucial.

### BRIEF SUMMARY

An embodiment of the present disclosure is a pressure sensor. The pressure sensor may comprise a flexible nanopaper, and a graphene film on one side of the flexible nanopaper.

In at least some embodiments, the nanopaper may be a water-resistant nanopaper.

In at least some embodiments, a thickness of the water-resistant nanopaper may be from 20 μm to 100 μm.

In at least some embodiments, the graphene film may comprise three to ten graphene sheets. Each of the three to ten graphene sheets may be a self-assembled layer of graphene powder.

In at least some embodiments, the graphene film may comprise one to three graphene sheets. Each of the one to three graphene sheets may be formed by deposition.

In at least some embodiments, the graphene film may have a square resistance of from 1,000Ω/□ to 30,000Ω/□.

In at least some embodiments, the graphene film may further comprise a pair of electrodes connected to different positions of the graphene film. In at least some embodiments, the pair of electrodes may be connected to two ends of the graphene film that are opposite to each other in a longitudinal direction of the graphene film.

In at least some embodiments, the pair of electrodes may be conductive copper tape or conductive silver wire.

Another embodiment of the present disclosure is a pressure detecting device. The pressure detecting device may comprise a pressure sensor as described above.

In at least some embodiments, the pressure detecting device may be configured to detect a pulse. In at least some embodiments, the pressure detecting device may be configured to detect a sound vibration.

In at least some embodiments, the pressure detecting device may further comprise a signal transmission module configured to transmit data acquired by the pressure sensor, and a pressure feedback module configured to display the data acquired by the pressure sensor.

Another embodiment of the present disclosure is a method of fabricating a pressure sensor. The pressure sensor may be as described above. The method may comprise: forming an ink layer by coating a graphene ink onto the nanopaper, the graphene ink having been formed by dispersing graphene powder in a solvent, and drying the ink layer to form the graphene film.

In at least some embodiments, the method may further comprise attaching a pair of electrodes to different positions of the graphene film.

In at least some embodiments, the graphene ink may contain 0.01% to 0.2% by mass of the graphene powder.

In at least some embodiments, a square resistance of the graphene film may be 1,000Ω/□ to 30,000Ω/□.

Another embodiment of the present disclosure is a method of detecting pressure. The method may comprise determining a variation in a resistance of the graphene film in a pressure sensor over a time period, the pressure sensor having been attached to a surface of a subject. The pressure sensor may be as described above. The method may further comprise determining a pressure in the subject based on the variation in the resistance of the graphene film over the time period.

In at least some embodiments, the determining of the variation in the resistance of the graphene film may comprise measuring a deformation in a surface of the graphene film in contact with the surface of the subject.

In at least some embodiments, the pressure sensor may be attached to a skin surface of a user. The method may further comprise determining a pulse of the user based on the determined pressure.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the present disclosure are apparent from the following detailed description taken in conjunction with the accompanying drawings in which:

FIG. 1 shows a schematic diagram of a device for detecting pressure according to an embodiment of the present disclosure;

FIG. 2 shows a schematic diagram of a pressure sensor according to an embodiment of the present disclosure;

FIG. 3 shows a photograph illustrating the water-resistance of a nanopaper in a pressure sensor according to the present disclosure;

FIG. 4 shows a schematic diagram illustrating a method of fabricating a pressure sensor according to an embodiment of the present disclosure;

FIG. 5 shows a schematic diagram illustrating a method of fabricating a pressure sensor according to another embodiment of the present disclosure;

FIG. 6 shows a flowchart of a method of fabricating a pressure sensor according to an embodiment of the present disclosure;

FIG. 7 shows a flowchart of a method of fabricating a pressure sensor according to another embodiment of the present disclosure;

FIG. 8 shows a schematic diagram of a pressure sensor according to an embodiment of the present disclosure in operation.

FIG. 9 shows a graph of change in resistance versus time based on heartbeat data obtained using a pressure sensor according to an embodiment of the present disclosure.

FIG. 10 shows a graph of change in resistance versus time based on heartbeat data obtained using a pressure sensor according to an embodiment of the present disclosure.

FIG. 11 shows a graph of change in resistance versus time based on sound data obtained using a pressure sensor according to an embodiment of the present disclosure.

The various features of the drawings are not to scale as the illustrations are for clarity in facilitating one skilled in the art in understanding the invention in conjunction with the detailed description.

### DETAILED DESCRIPTION

Next, the embodiments of the present disclosure will be described clearly and concretely in conjunction with the accompanying drawings, which are described briefly above. The subject matter of the present disclosure is described with specificity to meet statutory requirements. However, the description itself is not intended to limit the scope of this disclosure. Rather, the inventors contemplate that the claimed subject matter might also be embodied in other ways, to include different steps or elements similar to the ones described in this document, in conjunction with other present or future technologies.

While the present technology has been described in connection with the embodiments of the various figures, it is to be understood that other similar embodiments may be used or modifications and additions may be made to the described embodiments for performing the same function of the present technology without deviating therefrom. Therefore, the present technology should not be limited to any single embodiment, but rather should be construed in breadth and scope in accordance with the appended claims. In addition, all other embodiments obtained by one of ordinary skill in the art based on embodiments described in this document are considered to be within the scope of this disclosure.

A numerical range modified by “approximately” herein means that the upper and lower limits of the numerical range can vary by 10% thereof. A number modified by “approximately” herein means that the number can vary by 10% thereof.

Demand for pressure sensors have been gradually increasing for practical applications such as diagnostics and therapeutics. Small variations in pressure may carry great significance in many situations. For example, detecting variations in pulse during medical diagnosis can be symptomatic of certain medical conditions, or detecting variations in sound can help establish human-computer interaction platform. In situations such as those, the accurate detection of the small variations in pressure is crucial.

Conventional high-sensitivity pressure sensors are usually formed on synthetic polymer substrates as such polydimethyl siloxane (PDMS), polyethylene terephthalate (PET), or polyimide (PI). However, these conventional synthetic polymer materials do not decompose easily, and the disposal of those polymer materials pose serious environmental threat. In addition, the conventional synthetic polymer materials are hydrophobic, and tend to have poor gas permeability. When used in a sensor intended for detecting pulse, extended contact between the substrate of the sensor and the human skin can cause discomfort, and worse, allergic reactions. In other words, conventional synthetic polymer materials have poor biocompatibility. There is thus a need for a pressure sensor with improved biocompatibility and biodegradability.

Natural fibers have emerged in recent years as useful reinforcement in polymer composites because of their sustainability, renewability, biodegradability, low thermal expansion, manufacturer-friendly attributes such as low density and abrasiveness, excellent mechanical properties such as very high specific stiffness and strength and consumer-friendly attributes such as lower price and higher performance. Nanopaper is a film self-assembled from nanocellulose materials. For example, Chinese Patent Application No. 201810063040.9 discloses a water-resistant nanopaper. The nanopaper is composed of nanocellulose (the cellulose may also contain carboxyl groups) with polysaccharide molecules (such as starch, cellulose, chitin, and the like) adsorbed on the surface. The nanocellulose has a diameter of less than 100 nm, and more particularly, within the range of 10 nm to 50 nm. The nanopaper has a thickness of 30-100 μm, and a surface roughness of less than 10 nm.

Pressure Sensor

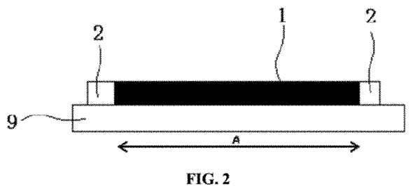

The present disclosure provides a pressures sensor. As shown in FIG. 2, the pressure sensor comprises a flexible nanopaper 9, and a graphene film 1 on a surface of the flexible nanopaper 9.

In the pressure sensor according to the present disclosure, the flexible nanopaper 9 is a substrate, and the graphene film 9 on the surface of the flexible nanopaper 9 is the sensing element.

Graphene is generally a monolayer of carbon atoms bound in a hexagonal honeycomb lattice. The carbon atoms in the monolayer have the same distribution pattern as the carbon atoms in a sheet of graphene. A film composed of graphene has excellent transparency and conductivity. In addition, the crack structure in the graphene film and the relative slip between graphene sheets contribute to the increased sensitivity of a graphene film in registering changes in resistance in response to sensed pressure. Even a small deformation in the graphene film (for example, at a magnitude of 0.1%) may be sufficient to induce a stable change in resistance. As such, in the pressure sensor according to the present disclosure, when the graphene film 1 is provided on the nanopaper 9, a small deformation (or vibration) due to a pressure change is transferred to the graphene film 1, which causes change in the resistance of the graphene film 1. Changes in the resistance of the graphene film 1 are highly sensitive to changes in pressure, and by measuring the change in resistance of the graphene film 1, the present disclosure makes it possible to improve the sensitivity of pressure detection.

In the pressure sensor according to the present disclosure, the flexible nanopaper 9 forms the substrate. Nanopaper 9 is composed of cellulose, and therefore biodegradable and environmentally friendly. In addition, the nanopaper 9 has a structure similar to that of a regular paper, and therefore, has excellent air permeability (breathability) and biocompatibility. Even after an extended contact, the risk of allergic reactions to a pressure sensor having a nanopaper substrate is significantly reduced as compared to one having a conventional synthetic polymer substrate.

In some embodiments, the nanopaper 9 is a water-resistant nanopaper, for example, as described in Chinese Patent Application No. 201810063040.9. Conventional nanopaper contains nanocellulose, which generally contains a large amount of hydroxyl groups. As a result, the nanopaper swells easily after absorbing water. Swelling puts stress on the surface of the nanopaper, and causes deformation in the nanopaper surface, which can in turn interfere with detection and may even cause breakage in the surface and device failure.

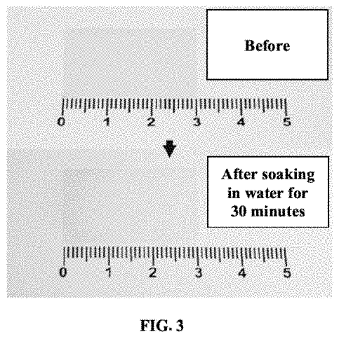

FIG. 3 shows a photograph illustrating the water-resistance of a nanopaper in a pressure sensor according to the present disclosure. In FIG. 3, the nanopaper is placed on a background paper. The top photograph in FIG. 3 shows a nanopaper before being soaked in water, and the bottom photograph in FIG. 3 shows the nanopaper after being soaked in water for 30 minutes. A comparison of the top and bottom photographs in FIG. 3 shows that the nanopaper in a pressure sensor according to the present disclosure does not swell or deform even after exposure to water. In other words, a property of the nanopaper is that it does not deform or swell after being exposed to moisture or water, so that the presence of moisture or water does not interfere with the detection functions of a pressure sensor containing the nanopaper as the substrate. In addition, water-resistant nanopaper is hydrophilic, so that it can be wetted and then directly affixed to the subject or object (for example, a human patient or an audio speaker) being examined. No other means of adherence are necessary to secure the pressure sensor, and the convenience of using the pressure sensor is increased significantly.

In some embodiments, the water-resistant nanopaper has a total thickness of 20 μm-100 μm.

When the nanopaper has a thickness within the above range, it can provide the pressure sensor with sufficient strength, while still allowing deformations in the sensing element (for example, the graphene film 1) to be transmitted with high sensitivity.

In some embodiments, the graphene film 1 comprises at least one layer of graphene sheet that is composed of self-assembled graphene powder. The graphene powder may be generally prepared from graphite. Further, the graphene powder may be prepared by any appropriate means known to a person of ordinary skill in the art, and in this regard, the present disclosure is not particularly limited.

In some embodiments, the graphene film 1 comprises a single layer of graphene sheet. In some embodiments, the graphene film 1 comprises a plurality of graphene sheets, and more particularly, the graphene film 1 may comprise 3 to 10 graphene sheets.

Graphene powder self-assemble into a larger, ordered three-dimensional sheet. The graphene film 1 may comprise different numbers of graphene sheets at different positions, but the number of graphene sheets at a given position in the graphene film 1 should be from 3 to 10.

In some embodiments, the graphene film 1 comprises one or more graphene sheets that are formed by growth. When the graphene sheets are formed by growth, the graphene film 1 may comprise at least one graphene sheet, and no more than three graphene sheets. In some embodiments, the graphene sheets are formed by chemical vapor deposition (CVD), during which between one and three graphene sheets are deposited to form the graphene film 1.

In some embodiments, the graphene sheets are formed by electrochemical exfoliation.

In some embodiments, the graphene film 1 has a square resistance of 1,000Ω/□ to 30,000Ω/□. In some embodiments, the square resistance of the graphene film 1 is no more than 20,000Ω/□. In some embodiments, the square resistance of the graphene film is at least 4000Ω/□.

The resistance of the graphene film 1 may be adjusted by adjusting the number of graphene sheets in the graphene film 1, which in turn adjusts the thickness of the graphene film 1. It has been found that when the square resistance of the graphene film 1 is within the above range, the accuracy of the pressure detections improves.

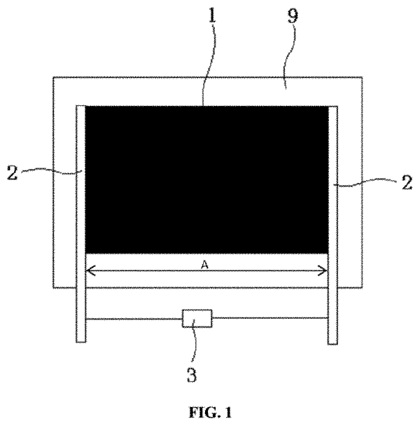

In some embodiments, the pressure sensor further comprises a pair of electrodes 2. The electrodes 2 connected to the graphene film 1 may be disposed directly in the pressure sensor, and configured to measure the resistance of the pressure sensor. The electrodes 2 are connected to different portions of the graphene film 1. For example, as shown in FIGS. 1 and 2, the graphene film 1 may have an elongated shape, and the electrodes 2 may be connected to two ends of the graphene film 1 that are opposite to each other in a longitudinal direction of the graphene film 1 (direction A in FIGS. 1 and 2). This configuration of the electrodes 2 may improve conductivity and the accuracy of the resistance measurements. The electrodes 2 are composed of conductive copper tape or conductive silver wire. More particularly, the electrodes 2 may be conductive copper tape adhered to the graphene film 1, or conductive silver wire fixed to the graphene film 1.

Pressure Detecting Device

The present disclosure provides a device for detecting pressure. The pressure detecting device comprises a pressure sensor as described above. The pressure detecting device may further comprise a resistance detecting unit 3 that is connected to the pressure sensor and is configured to measure the resistance between two different positions in the graphene film 1 of the pressure sensor.

The resistance detecting unit 3 is configured to measure the resistance of the graphene film 1, and as shown in FIG. 1, is connected to the pressure sensor to form the pressure detecting device. In some embodiments, the resistance detecting unit 3 is a resistance meter. Since the resistance measured by the resistance detecting unit 3 correlates with pressure, the pressure detecting device of the present disclosure is configured to measure pressure.

In some embodiments, the pressure detecting device may comprise a signal transmission module and a pressure feedback module. The signal transmission module may be a circuit configured to transmit data acquired by the pressure sensor, including but not limited to data relating to pressure measurements. The design, construction, and configuration of the signal transmission module are not particularly limited, and may be any appropriate design, construction, and/or configuration known to a person of ordinary skill in the art. For example, in some embodiments where the pressure detecting device is configured to detect a pulse of a human subject, the signal transmission module may be a circuit configured to transmit data relating to variations in the resistance of the graphene film due to the human subject's pulse. In some embodiments where the pressure detecting device is configured to detect a sound vibration, the signal transmission module may be a circuit configured to transmit data relating to variations in the resistance of the graphene film due to soundwaves emitted by a sound source. The pressure feedback module may be a display unit configured to display to the user the data acquired by the pressure sensor, including but not limited to data relating to pressure measurements. The design, construction, and configuration of the pressure feedback module are not particularly limited, and may be any appropriate design, construction, and/or configuration known to a person of ordinary skill in the art.

The pressure detecting device may comprise additional components, for example, a controller or CPU configured to convert the measured resistance value into a pressure value, and an output unit (for example, a display unit) configured to display the measured resistance and the calculated pressure. It is understood that additional components and/or accessories may be provided within a pressure detecting device of the present disclosure without departing from the spirit and scope of the present disclosure. A person of ordinary skill in the art would readily appreciate that the configuration of a pressure detecting device is not limited to the embodiments shown in the figures, and a pressure detecting device may include any additional components that are typically found in a pressure detecting device and/or that are provided according to any particular purpose for which the pressure detecting device is intended.

In some embodiments, for example, as shown in FIG. 1, the resistance detecting unit 3 is between and connected to the pair of electrodes 2 of the pressure sensor, and is configured to measure the resistance between the pair of electrodes 2.

In some embodiments, the pressure sensor does not comprise the pair of electrodes 2. In that case, the resistance detecting unit 3 may comprise probe, clip, and the like for connecting the resistance detecting unit 3 to the graphene film 1 at two different positions in or on the graphene film 1.

In some embodiments, the pressure detecting device may not comprise a resistance detecting unit 3. The pressure sensor may instead be connected to a resistance meter external to the pressure detecting device. The external resistance meter is then configured to measure resistance, and to achieve the pressure detecting functions.

Method of Detecting Pressure

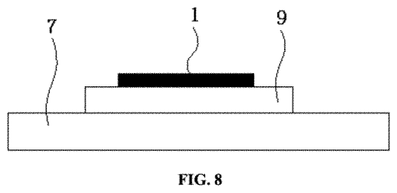

The present disclosure provides a method of detecting pressure. As shown in FIG. 8, the nanopaper 9 of the pressure sensor is attached to the subject 7 to be tested. More particularly, the nanopaper 9 is attached to the subject 7 via the surface of the nanopaper 9 without the graphene film 1. In FIG. 8, the surface of the nanopaper 9 opposite from that bearing the graphene film 1 is contact with the subject 7.

The nanopaper 9 may be attached to the subject 7 by any appropriate means known to a person of ordinary skill in the art, including, but not limited to, adhesive tape, so long as the means of attachment allows pressure-related deformations in the surface of the subject 7 to be transmitted to the graphene film 1 of the pressure sensor.

The method of detecting pressure according to the present disclosure comprises determining a variation in a resistance of the graphene film 1 in the pressure sensor according to claim 1 over a time period. Resistance of the graphene film 1 is thus acquired. More particularly, the determining of the variation in the resistance of the graphene film 1 may comprise measuring a deformation in a surface of the graphene film in contact with the surface of the subject. The pressure in the subject 7 being examined is then determined based on the variation in the resistance of the graphene film 1 over the time period.

In embodiments where the nanopaper 9 is water-resistant nanopaper, the surface of the water-resistant nanopaper 9 without the graphene film 1 is wetted, and then adhered to the subject 7 being examined. Water-resistant nanopaper is hydrophilic, so that it can be wetted and then directly affixed to the subject or object (for example, a human patient or an audio speaker) being examined. No other means of adherence are necessary to secure the pressure sensor, and the convenience of using the pressure sensor is increased significantly. In addition, the water-resistant nanopaper detaches automatically when the wetted surface dries.

In some embodiments, the method of detecting pressure is for detecting sound. The subject 7 to be examined is the source of sound, for example, an audio speaker. The pressure sensor according to the present disclosure is attached on the sound source to detect soundwaves being emitted by the sound source, and the measurements can be used to establish human-computer interaction platform.

In some embodiments, the method of detecting pressure is for detecting a human pulse. The pressure sensor of the present disclosure may be used in any appropriate manner known to a person of ordinary skill in the art to measure the pulse of a human subject.

Wearable Pressure Detection Device

The present disclosure provides a wearable pressure detection device. The wearable pressure detection device comprises a pressure sensor as described above.

The pressure sensor according to the present disclosure may be incorporated in a wearable pulse detection device. The wearable pulse detection device may be a device that can be won on a human body (for example, by adhesion to a wetted surface on the wrist) in order to detect the pulse of the human subject. The use of the pressure sensor according to the present disclosure improves accuracy of the detection results and the sensitivity of the detection device. The pressure sensor according to the present disclosure is user-friendly, convenient, and biocompatible to reduce the risk of allergic reactions. In addition, the pressure sensor according to the present disclosure utilizes a nanopaper as a substrate, so that the pressure sensor is biodegradable and environmentally friendly.

It is understood that additional components and/or accessories may be provided within a wearable pressure detection device of the present disclosure without departing from the spirit and scope of the present disclosure. A person of ordinary skill in the art would readily appreciate that the configuration of a wearable pressure detection device is not limited to the embodiments shown in the figures, and a wearable pressure detection device may include any additional components that are typically found in a wearable pressure detection device and/or that are provided according to any particular purpose for which wearable pressure detection device is intended.

For example, the wearable pressure detection device may additionally comprise a removable protective layer that encapsulates the pressure sensor to protect the cleanness of the pressure sensor prior to use.

In some embodiments, the wearable pressure detection device comprises a resistance detecting unit 3. The resistance detecting unit 3 is connected to the pressure sensor and is configured to measure the resistance between two different positions in the graphene film 1 of the pressure sensor.

The resistance detecting unit 3 is configured to measure the resistance of the graphene film 1, and as shown in FIG. 1, is connected to the pressure sensor to form the pressure detecting device. In some embodiments, the resistance detecting unit 3 is a resistance meter. Since the resistance detecting unit 3 is incorporated into the wearable pressure detection device, the wearable pressure detection device can directly acquire the pressure values for the human subject (via the resistance values acquired by the resistance detecting unit 3).

In some embodiments, the pressure sensor does not comprise the pair of electrodes 2. In that case, the resistance detecting unit 3 may comprise probe, clip, and the like for connecting the resistance detecting unit 3 to the graphene film 1 at two different positions in or on the graphene film 1.

In some embodiments, the pressure detecting device may not comprise a resistance detecting unit 3. The pressure sensor may instead be connected to a resistance meter external to the pressure detecting device. The external resistance meter is then configured to measure resistance, and to achieve the pressure detecting functions.

Method of Fabricating Pressure Sensor

The present disclosure provides a method of fabricating a pressure sensor.

Generally, the method comprises providing the graphene film 1 on a surface of the nanopaper 1. The graphene film 1 may be provided on the nanopaper 1 by any appropriate means known to a person of ordinary skill in the art, and in this regard, the present disclosure is not particularly limited.

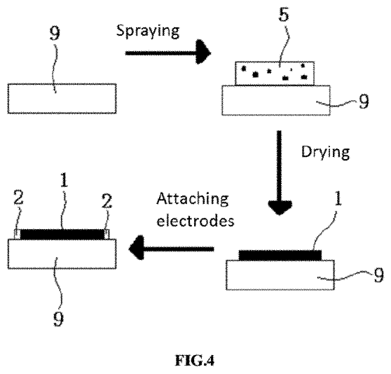

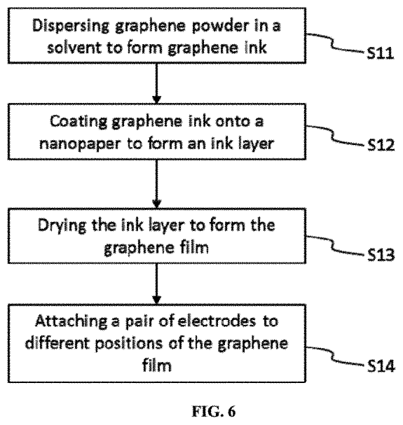

FIG. 4 shows a schematic diagram illustrating a method of fabricating a pressure sensor according to an embodiment of the present disclosure. FIG. 6 shows a flowchart of a method of fabricating a pressure sensor according to an embodiment of the present disclosure.

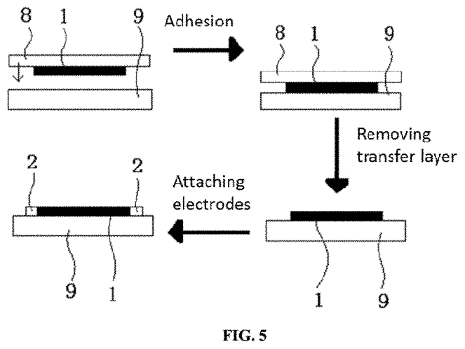

As shown in FIGS. 4 and 5, the method comprises the following steps:

In step S11, graphene powder is dissolved in a solvent to forma graphene ink. More particularly, a large amount of graphene powder is uniformly dispersed in a solvent to form a graphene-containing ink.

In some embodiments, the solvent is water or ethanol. The graphene ink contains 0.01% to 0.2% by mass of graphene powder. In some embodiments, the graphene ink contains 0.1% by weight of graphene powder. These configurations of the solvent and graphene powder help ensure uniform and stable dispersion of graphene powder in the graphene ink.

In step S12, the graphene ink is coated onto the nanopaper 9 to form an ink layer 5.

Due to the composition of the graphene ink, the ink layer 5 contains a large amount of dispersed graphene powder.

In some embodiments, the graphene ink is coated by spraying. The square resistance of final graphene film 1 depends on the thickness of the graphene film, that is, the number of graphene sheets forming the graphene film 1. As described above, in some embodiments, the graphene film 1 comprises a single layer of graphene sheet, and in some embodiments, the graphene film 1 comprises a plurality of graphene sheets, and more particularly, the graphene film 1 may comprise 3 to 10 graphene sheets. The number of graphene sheets forming the graphene film 1 in turn may be controlled by the amount of graphene powder. The amount of graphene powder applied to the surface of the nanopaper 9 may be controlled, for example, by controlling the concentration of the graphene powder in the graphene ink, the spraying rate, the spraying time, and the like, so as to control the square resistance of the graphene film 1 to within the range of 1,000Ω/□ to 30,000Ω/□.

However, the present disclosure does not particularly limit the manner in which the graphene ink is coated onto the nanopaper, and the graphene ink may be coated by any appropriate means known to a person of ordinary skill in the art, so long as the resulting graphene film exhibits a square resistance within the range of 1,000Ω/□ to 30,000Ω/□.

In step S13, the ink layer 5 is dried to remove the solvent, and the graphene powder in the ink layer 5 is allowed to self-assemble into the graphene film 1.

During the drying process, the solvent in the ink layer 5 evaporates. Since the graphene powder has a lamellar structure, evaporation of the solvent deposits the graphene powder onto the nanopaper 9, and the graphene powder self-assembles into the graphene film 1.

In step S14, a pair of electrodes 2 are connected to the graphene film 1.

The electrodes 2 may be composed of conductive copper tape or conductive silver wire. Each of the electrodes 2 is connected to a different position on the graphene film. For example, the electrodes 2 may be connected to opposite ends of the graphene film 1, as shown in FIGS. 1 and 2.

After the graphene film 1 is formed in step S13, the electrodes 2 are connected to the graphene 1. In some embodiments, the electrodes 2 are conductive copper tapes that are adhered to different positions on the graphene film 1.

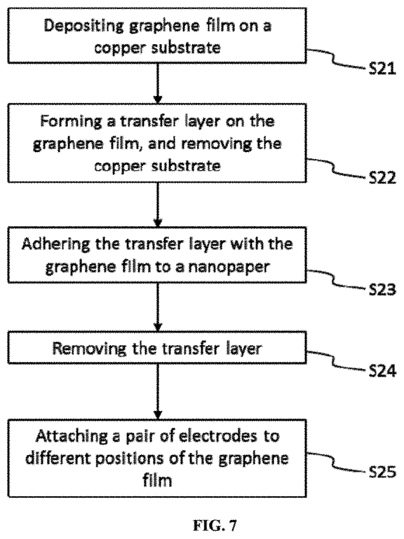

FIG. 5 shows a schematic diagram illustrating a method of fabricating a pressure sensor according to another embodiment of the present disclosure. FIG. 7 shows a flowchart of a method of fabricating a pressure sensor according to another embodiment of the present disclosure.

As shown in FIGS. 5 and 7, the graphene film 1 is formed on a transfer layer 8. The transfer layer 8 is provided on a surface of the nanopaper 9, so that the graphene film 1 is in contact with the nanopaper 9. The transfer layer 8 is removed, and the graphene film 1 remains on the nanopaper 9.

In the embodiments shown in FIGS. 5 and 7, the graphene film 1 is formed separately on the transfer layer 8, and then transferred onto the nanopaper 9. The process of forming and transferring the graphene film may be as described in Li, et al., “Large-area synthesis of high-quality and uniform graphene films on copper foils”, Science, Vol. 324, pp. 1312-4 (Jun. 5, 2009). In some embodiments, the process of forming and transferring the graphene film may be as follows:

In step S21, a graphene film 1 is formed, for example, by chemical vapor deposition, on a copper substrate. The graphene may comprise one or more sheets of graphene.

In step S22, a transfer layer 8 is formed on the graphene film 1. In some embodiments, the transfer layer 8 is composed of a (meth)acrylate polymer, for example, polymethyl methacrylate (PMMA). The copper substrate is then removed, for example, by chemical corrosion, in order to transfer the graphene film 1 onto the transfer layer 8.

In step S23, the transfer layer 8 is adhered to the nanopaper 9 in a manner so that the graphene film 1 is sandwiched between the transfer layer 8 and the nanopaper 9.

In step S24, the transfer layer 8 is dissolved using acetone, and the graphene film 1 is transferred to the nanopaper 9.

In step S25, a pair of electrodes 2 are connected to the graphene film 1.

The electrodes 2 may be composed of conductive copper tape or conductive silver wire. Each of the electrodes 2 is connected to a different position on the graphene film. For example, the electrodes 2 may be connected to opposite ends of the graphene film 1, as shown in FIG. 5.

### EXAMPLES

### Example 1

A graphene ink containing 0.1% by weight of graphene powder is sprayed for 10 seconds onto a 20-μm nanopaper to form an ink layer. The ink layer is allowed to air dry, and the graphene powder is observed to self-assemble on the nanopaper into a graphene film. A pair of conductive copper tapes are affixed onto opposite ends of the graphene film to form electrodes. Measurement using a multimeter indicates that the graphene film has a square resistance of 20,000Ω/□. A pressure sensor according to the present disclosure is thus formed.

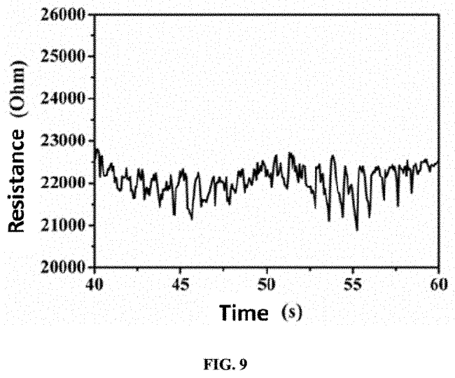

To attach the pressure sensor to the wrist of a human patient, the wrist is wetted slightly to allow the nanopaper substrate of the pressure sensor to adhere to the skin via capillary action (hygroscopy). A KEITHLEY® 4200 Semiconductor Characterization System is set to resistance mode, and the pair of electrodes of the pressure sensor are designated the source and drain electrodes, respectively. Resistance between the two electrodes is measured in real time, and the variation of resistance with time is recorded. The resistance (pressure) changes caused by the patient's heartbeat are detected. The result is shown in FIG. 9.

As shown in FIG. 9, each beat of the heart causes a pressure change on the pressure sensor, which registers as a change in the resistance between the two electrodes. FIG. 9 shows that the pressure sensor according to the present disclosure can be used to accurately monitor pulse.

### Example 2

A graphene ink containing 0.1% by weight of graphene powder is sprayed for 20 seconds onto a 100-μm nanopaper to form an ink layer. The ink layer is allowed to air dry, and the graphene powder is observed to self-assemble on the nanopaper into a graphene film. A pair of conductive copper tapes are affixed onto opposite ends of the graphene film to form electrodes. Measurement using a multimeter indicates that the graphene film has a square resistance of 1,200Ω/□. A pressure sensor according to the present disclosure is thus formed.

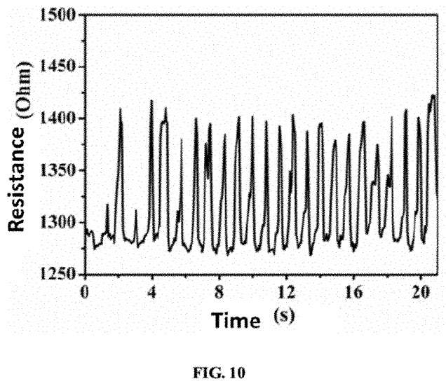

To attach the pressure sensor to the wrist of a human patient, the wrist is wetted slightly to allow the nanopaper substrate of the pressure sensor to adhere to the skin via capillary action (hygroscopy). A KEITHLEY®4200 Semiconductor Characterization System is set to resistance mode, and the pair of electrodes of the pressure sensor are designated the source and drain electrodes, respectively. Resistance between the two electrodes is measured in real time, and the variation of resistance with time is recorded. The resistance changes caused by the patient's heartbeat are detected. The result is shown in FIG. 10.

As shown in FIG. 10, each beat of the heart causes a pressure change on the pressure sensor, which registers as a change in the resistance between the two electrodes. FIG. 10 shows that the pressure sensor according to the present disclosure can be used to accurately monitor pulse.

### Example 3

A graphene ink containing 0.1% by weight of graphene powder is sprayed for 15 seconds onto a 100-μm nanopaper to form an ink layer. The ink layer is allowed to air dry, and the graphene powder is observed to self-assemble on the nanopaper into a graphene film. A pair of conductive copper tapes are affixed onto opposite ends of the graphene film to form electrodes. Measurement using a multimeter indicates that the graphene film has a square resistance of 4,000Ω/□. A pressure sensor according to the present disclosure is thus formed.

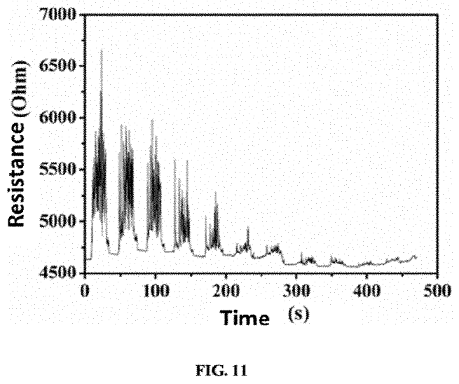

The pressure sensor is affixed to an audio speaker and secured by tape. A KEITHLEY® 4200 Semiconductor Characterization System is set to resistance mode, and the pair of electrodes of the pressure sensor are designated the source and drain electrodes, respectively. The volume on the speaker is reduced incrementally, and the corresponding changes in the resistance between the electrodes are measured in real time. Each change in volume causes a change in pressure on the pressure sensor, which registers as a change in the resistance between the electrodes. The changes in resistance are measured in real time. The results are shown in FIG. 11.

As shown in FIG. 11, the pressure sensor according to the present disclosure can accurately detect changes in pressure caused by sound.

### Example 4

A graphene ink containing 0.1% by weight of graphene powder is deposited, via chemical vapor deposit, onto a transfer layer composed of polymethyl methacrylate to form the graphene film. The transfer layer bearing the graphene film is then adhered to a 30-μm nanopaper. Acetone is applied to the layered structure to dissolve the transfer layer. A pair of conductive copper tapes are affixed onto opposite ends of the graphene film to form electrodes. Measurement using a multimeter indicates that the graphene film has a square resistance of 1,000Ω/□. A pressure sensor according to the present disclosure is thus formed.

In the description of the specification, references made to the term “some embodiment,” “some embodiments,” and “exemplary embodiments,” “example,” and “specific example,” or “some examples” and the like we intended to refer that specific features and structures, materials or characteristics described in connection with the embodiment or example that are included in at least some embodiments or example of the present disclosure. The schematic expression of the terms does not necessarily refer to the same embodiment or example. Moreover, the specific features, structures, materials or characteristics described may be included in any suitable manner in any one or more embodiments or examples. In addition, for a person of ordinary skill in the art, the disclosure relates to the scope of the present disclosure, and the technical scheme is not limited to the specific combination of the technical features, and also should covered other technical schemes which are formed by combining the technical features or the equivalent features of the technical features without departing from the inventive concept. What is more, the terms “first” and “second” are for illustration purposes only and are not to be construed as indicating or implying relative importance or implied reference to the quantity of indicated technical features. Thus, features defined by the terms “first” and “second” may explicitly or implicitly include one or more of the features. In the description of the present disclosure, the meaning of “plural” is two or more unless otherwise specifically and specifically defined.

The principle and the embodiment of the present disclosures are set forth in the specification. The description of the embodiments of the present disclosure is only used to help understand the method of the present disclosure and the core idea thereof. Meanwhile, for a person of ordinary skill in the art, the disclosure relates to the scope of the disclosure, and the technical scheme is not limited to the specific combination of the technical features, and also should covered other technical schemes which are formed by combining the technical features or the equivalent features of the technical features without departing from the inventive concept. For example, technical scheme may be obtained by replacing the features described above as disclosed in this disclosure (but not limited to) with similar features.

1. A pressure sensor, comprising:
a flexible nanopaper, and
a graphene film on one side of the flexible nanopaper.

2. The pressure sensor according to claim 1,
wherein the nanopaper is a water-resistant nanopaper.

3. The pressure sensor according to claim 2,
wherein a thickness of the water-resistant nanopaper is from 20 μm to 100 μm.

4. The pressure sensor according to claim 1,
wherein the graphene film comprises three to ten graphene sheets, each of the three to ten graphene sheets being a self-assembled layer of graphene powder.

5. The pressure sensor according to claim 1,
wherein the graphene film comprises one to three graphene sheets, each of the one to three graphene sheets being formed by deposition.

6. The pressure sensor according to claim 1,
wherein the graphene film has a square resistance of from 1,000Ω/□ to 30,000Ω/□.

7. The pressure sensor according to claim 1, further comprising a pair of electrodes connected to different positions of the graphene film.

8. The pressure sensor according to claim 7,
wherein the pair of electrodes are connected to two ends of the graphene film that are opposite to each other in a longitudinal direction of the graphene film.

9. The pressure sensor according to claim 7,
wherein the pair of electrodes are conductive copper tape or conductive silver wire.

10. A pressure detecting device, comprising the pressure sensor according to claim 1.

11. The pressure detecting device according to claim 10, wherein the pressure detecting device is configured to detect a pulse.

12. The pressure detecting device according to claim 10, wherein the pressure detecting device is configured to detect a sound vibration.

13. The pressure detecting device according to claim 10, further comprising a signal transmission module configured to transmit data acquired by the pressure sensor, and a pressure feedback module configured to display the data acquired by the pressure sensor.

14. A method of fabricating the pressure sensor according to claim 1, the method comprising:
forming an ink layer by coating a graphene ink onto the nanopaper, the graphene ink having been formed by dispersing graphene powder in a solvent, and
drying the ink layer to form the graphene film.

15. The method according to claim 14, further comprising attaching a pair of electrodes to different positions of the graphene film.

16. The method according to claim 14,
wherein the graphene ink contains 0.01% to 0.2% by mass of the graphene powder.

17. The method according to claim 14,
wherein a square resistance of the graphene film is 1,000Ω/□ to 30,000Ω/□.

18. A method of detecting pressure, the method comprising:
determining a variation in a resistance of the graphene film in the pressure sensor according to claim 1 over a time period, the pressure sensor having been attached to a surface of a subject, and
determining a pressure in the subject based on the variation in the resistance of the graphene film over the time period.

19. The method according to claim 18, wherein the determining of the variation in the resistance of the graphene film comprises measuring a deformation in a surface of the graphene film in contact with the surface of the subject.

20. The method according to claim 18,
wherein the pressure sensor is attached to a skin surface of a user, and wherein the method further comprises determining a pulse of the user based on the determined pressure.
